(19) United States
(12) Patent Application Publication
(10) Pub. No.: US 2016/0281171 A1
(43) Pub. Date: Sep. 29, 2016

(54) TARGETED SCREENING FOR MUTATIONS

(71) Applicant: INVIVOSCRIBE TECHNOLOGIES, INC., San Diego, CA (US)

(72) Inventors: Jeffrey E. Miller, San Diego, CA (US); Brad Patay, San Diego, CA (US); Andrew Carson, San Diego, CA (US); Suzanne Graham, San Diego, CA (US)

(21) Appl. No.: 15/034,840

(22) PCT Filed: Nov. 6, 2014
(86) PCT No.: PCT/US14/64438

§ 371 Date: May 5, 2016

Related U.S. Application Data
(60) Provisional application No. 61/900,728, filed on Nov. 6, 2013.

(51) Int. Cl. C12Q 1/68

(52) U.S. Cl. CPC C12Q 1/6886; C12Q 2600/156

(57) ABSTRACT

Compositions, methods and kits for genomic screening, genetic analysis, and gene discovery. In some embodiments the disclosed methods can detect large internal tandem duplications, or novel translocations, as well as identify the genomic breakpoint of novel translocations when only one of the two fusion partners is known or targeted. This is accomplished by employing a series of carefully selected capture probes to target genome-specific and disease-specific areas of target genes that harbor disease related somatic mutations, insertions/deletions or are involved in translocations.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57, including U.S. Patent Application 61/900,728, filed on Nov. 6, 2013.

### BACKGROUND OF THE INVENTION

1. Field of the Invention

Provided herein is technology relating to genotyping, specifically a sample preparation, sequencing and bioinformatics strategy for identifying mutations/variants, including single nucleotide variants, insertions, deletions and structural variants such as translocations present in an biological sample, preferably a sample containing cancer cells.

2. Description of the Related Art

Traditionally, diagnosis of disease has relied primarily on morphological examination and symptom presentation. However, using this approach, diagnosis is possible only after the disease has progressed to the point of physical manifestation. For many diseases, early detection can lead to early treatment, significantly improving recovery and survival rates. Furthermore, detection of susceptibility or propensity for a disease prior to the appearance of symptoms will maximize awareness and enable changes in lifestyle, which can delay disease onset, minimize the severity of the disease, or prevent the disease state from occurring altogether. The discovery of mutations that determine phenotypes is a fundamental premise of genetic research. Over the past several years, there has been considerable interest in the development of analytical tools and methods to probe nucleic acid sequences for information to aid in the prevention, early detection, diagnosis, stratification, monitoring, and treatment of disease.

However, the vast amount of data encoded in nucleic acid sequences and the high cost of sequencing have stymied the practical utility of, for example, whole genome sequencing and analysis of mutations that are associated with disease. These efforts have been further complicated and are particularly problematic when somatic mutations play a role in disease etiology. Currently, diagnostic laboratories routinely perform screening to identify the most important, clinically actionable mutations. However, existing tests and sequencing technologies are limited by; 1) the cost of designing, validating and performing multiple individual assays (each of which adds both time and incremental cost to diagnostic assessment or workup) and, 2) the clinical sensitivity, which makes current tests unsuitable both for detecting somatic mutations in heterogeneous cell populations (a characteristic of malignancies) and in monitoring residual disease. Identifying all of the clinically relevant somatic mutations that exist at diagnosis, including mutations that may exist in small numbers or a subpopulation of cancer cells, continues to be a challenge for current test methods.

Monitoring minimal residual disease (MRD) is also a critical component of cancer treatment. MRD refers to the small numbers of neoplastic cells that survive in a cancer patient through the entire course of disease, most especially following treatment, when the patient is in cytogenetic or molecular remission. A very small number of such cells can cause relapse of the cancer, so the sensitivity of MRD detection is important in all aspects of treatment. For example, MRD can track the responsiveness of a particular patient to a particular therapy, serve as a basis for comparing different therapies, and provide information as to whether the cancer is in the initial stages of recurrence or relapse. However, accurate, sensitive and timely detection of the range of complex mutations that serve as biomarker candidates for MRD detection, particularly somatic mutations present in varying numbers in the diverse cell subpopulations characteristic of malignancies, has been a major obstacle to effective monitoring of patients during the course of their disease. Translocations, particularly those involving unknown fusion partners, are particularly resistant to identification using existing test methods.

In addition, current tests, even tests that use conventional molecular methods to identify mutations in individual biomarkers, do not interrogate the majority of hotspot mutations in the large number of genes that can affect patient outcome. In order to identify low frequency somatic mutations and interrogate the large number of genes that are driver mutations in cancer, new testing methods need to be developed and validated that utilize more efficient and sensitive technologies. These technologies and approaches could help keep pace, both with physician demands to optimize clinical care, and translational studies in support of drug development.

In order to maximize the value of these new tests and provide both optimized, personalized treatments and optimal enrollment in clinical trials, patient- and clone-specific ultra-sensitive personalized biomarker tests, developed in response to data generated from these new testing methods, also need to be developed in parallel so healthcare providers can effectively monitor and track the specific clones or subclones identified and associated with the disease.

The current ‘gold standard’ for nucleic acid sequencing, Sanger sequencing, has remained technologically static since its inception in the 1970s. The Sanger method uses DNA polymerase to synthesize a strand of DNA complementary to the target strand in the presence of 2′-deoxynucleotides (dNTPs) and 2′,3′-dideoxynucleotides (ddNTPs). The latter are irreversible DNA synthesis terminators, so sequencing is terminated whenever a ddNTP is added to the end of the growing oligonucleotide chain. This results in truncated oligonucleotides of varying lengths, each with a ddNTP at the 3′ end. These products are separated by size, and the pattern of ddNTP incorporation is used to elucidate the sequence of the original DNA strand.

This method initially required four reactions per template, one for each nucleobase found in DNA. Subsequent advances allowed combining the four ddNTPs together followed by fluorescent detection and identification of the different ddNTPs. Further advances have replaced the original polyacrylamide gel separation with capillary arrays and new separation polymers, which increased Sanger sequencing efficiency. These improvements provide a relatively low error rate and long read length.

However, this methodology is still relatively expensive, particularly for large sequencing projects. Far more importantly, Sanger sequencing is incapable of detecting mutations in a background of non-mutant templates, as the sequencing signals generated are from the pool of templates sequenced. This limitation requires that for detection, mutations must be present in more than 10-20% of the pooled templates molecules. Recent advances in next-generation sequencing (NGS), sometimes also referred to as massively parallel sequencing, have overcome this hurdle by enabling the collection of large amounts of sequence data from individual members of a library of template molecules, and this can be done at relatively low cost, as millions of individual sequencing reactions can be performed simultaneously.

NGS technologies utilize a number of different approaches to accomplish the simultaneous sequencing of individual templates. Just a few of the numerous examples include: emulsion polymerase chain reaction (PCR), attaching ssDNA fragments to a solid surface and conducting bridge amplification of single-molecule DNA templates, and using transposition through engineered single nanopore substrates to generate sequence information.

Next generation sequencing (NGS) technologies has started to facilitate whole-genome and focused discovery, which are critical components to a deeper understanding of, and ability to treat, genetically driven disorders. NGS is particularly important for addressing genetically driven disease states that have proven intractable to traditional genotypic analysis, whether due to the current limitations in mutation detection, lack of information processing capability, cost, or throughput. Some disorders, such as acute myeloid leukemia (AML), have proven particularly problematic for genotypic analysis due to the large number of important but complex and infrequent somatic mutations.

For example, AML is characterized by an increased number of myeloid cells in bone marrow and a concomitant arrest in cell maturation. The Cancer Genome Atlas (TCGA) Consortium completed a systematic survey of de novo AML, that is, AML not associated with previous therapy. The TCGA survey revealed most of the common recurrent somatic mutations. Despite the TCGA's modest sample size, a majority of common nonsynomous mutations were elucidated, because de novo AML has a low somatic mutation rate. Nonsynomous mutations are those that affect the amino acid sequence of a protein and therefore may exert a biological effect and are subject to selection. Thus, while minimal residual disease (MRD) monitoring has been used with success to evaluate and track the disease status of some leukemic patients, it has been difficult to both identify and monitor subsets of somatic mutations in leukemia due to the limited availability of assays that can monitor the myriad of possible somatic mutations at the sensitivity required.

Most AML cases are initiated in a single founding cell that evolves to several related subclones that harbor different somatic mutations. Although conventional diagnostic methods fail to reveal mutations in cryptic subclones these mutations often become the dominant clone at the time of leukemia relapse. In the United States, more than 14,000 individuals are newly diagnosed with the AML each year and many will succumb to this disease. Diagnostic assays are needed to help individuals enter into clinical trials that stratify patients for clinical trials based on clonal somatic mutations to utilize novel personalized therapeutics that could improve their outcome. FLT3 (FMS-related tyrosine kinase 3) targeted therapies, many of which are currently in phase II and phase III clinical trials, are examples of progress in this area. Furthermore, the diagnostic assays currently used to fully characterize AML require a number of different technologies that generally require testing different sample types or require splitting samples to ensure comprehensive testing. Turnaround times and costs can be prohibitive and impact patient care.

In addition to molecular diagnostic methods to support clinical treatment, precise characterization of the range of possible mutations in specific somatic mutations implicated in AML is required. For example, immortalized FMS-related tyrosine kinase 3 (FLT3) mutant cell lines that arise spontaneously and cell lines engineered to incorporate recurrent driver mutations will be needed to assist in clinical diagnostic and therapeutic translation, including development and validation of companion diagnostics. In FLT3, two major classes of variants in the FLT3 gene drive cytogenetically normal acute myeloid leukemia (AML): nonsynonymous somatic mutations, predominantly in the tyrosine kinase domains (TKD1 and TKD2), and somatic internal tandem duplications (ITD) in and around the juxtamembrane domain (JMD).

### SUMMARY OF THE INVENTION

An embodiment of the disclosed invention is a method of screening a nucleic acid sample for mutations comprising: (a) obtaining a nucleic acid sample; (b) fragmenting the nucleic acid sample; (c) contacting the fragmented nucleic acid sample with a panel of capture probes, wherein the panel of capture probes specifically capture targeted nucleic acid fragments which are identified as having or likely having a mutation; (d) isolating the targeted nucleic acid fragments captured by the panel of capture probes; (e) sequencing the isolated targeted nucleic acid fragments; and (f) analyzing the sequences of the isolated targeted nucleic acid fragments to identify mutations with prognostic and/or therapeutic significance.

An embodiment of the disclosed invention is a panel of nucleic acid capture probes comprising a plurality of nucleic acids, wherein the nucleic acids are 20-200 nucleotides in length, wherein the nucleic acids comprise at least 1,000 unique nucleic acid sequences, and wherein the nucleic acid sequences are complementary to target nucleic acids that are identified as having or likely having a mutation.

In any or all of the embodiments, the method further comprises: (b′) adding adaptor nucleic acids to the fragmented nucleic acids. In any or all of the embodiments the panel of capture probes comprise a plurality of nucleic acids comprising at least 1,000 unique nucleic acid sequences, at least 10,000 unique nucleic acid sequences, at least 100,000 unique nucleic acid sequences, at least 150,000 unique nucleic acid sequences, or at least 200,000 unique nucleic acid sequences. In any or all of the embodiments, the nucleic acid capture probes are 20-200 nucleotides in length, or 50-200 nucleotides in length, or 20-150 nucleotides in length. In any or all of the embodiments the nucleic acid capture probes have a nucleic acid sequence which is complementary to the targeted nucleic acid fragments, wherein the complementarity is at least 80% complementarity, 90% complementarity, 95% complementarity, or 100% complementarity. In any or all of the embodiments the method, further comprises: (b″) selecting the nucleic acid fragments to select nucleic acid fragments of 100-5,000 nucleotides in length, 200-1400 nucleotides in length, or 300-900 nucleotides in length, or 300-700 nucleotides in length. In any or all of the embodiments the isolated targeted nucleic acid fragments have an average length of 100-5,000 nucleotides in length, 200-1400 nucleotides in length, or 300-900 nucleotides in length, or 300-700 nucleotides in length. In any or all of the embodiments the sequencing of the isolated target nucleic acid fragments is at a read depth of at least 500×, at least 1000×, at least 10,000×, or at least 100,000×. In any or all of the embodiments the average length of the sequence reads of the isolated target nucleic acid fragments is at least 500 nucleotides, or at least 600 nucleotides, at least 700 nucleotides, or at least 1,000 nucleotides. In any or all of the embodiments the analyzing comprises aligning the sequences of the isolated targeted nucleic acid fragments to a reference sequence. In any or all of the embodiments the nucleic acid sample is isolated from a biological sample. In any or all of the embodiments the nucleic acid sample is isolated from a sample comprising cancer cells. In any or all of the embodiments the target nucleic acids are from genes identified as having a mutation in a cancer cell. In any or all of the embodiments the target nucleic acids are from genes identified in a public database as having a mutation in a cancer cell. In any or all of the embodiments the identified mutation is used for diagnostic, prognostic, or treatment purposes. In any or all of the embodiments the sample is from a patient, and the identified mutation is used for diagnostic, prognostic, or treatment purposes. In any or all of the embodiments the mutation is selected from the group consisting of a single nucleotide variant, an insertion, a deletion or a translocation. In any or all of the embodiments step (b′) is before step (c), or step (b′) is after step (c). In any or all of the embodiments (b″) is before step (c) or step (b″) is after step (c). In any or all of the embodiments the mutation is selected from the group consisting of a single nucleotide variant, an insertion, a deletion or a translocation. In any or all of the embodiments the target nucleic acids are from genes identified as having a mutation in a cancer cell. In any or all of the embodiments the target nucleic acids are from genes identified in a public database as having a mutation in a cancer cell. In any or all of the embodiments the panel of capture probes comprise at least 10,000 unique nucleic acid sequences complementary to at least 30 genes selected from Table 1. In any or all of the embodiments the cancer is AML.

### BRIEF DESCRIPTION OF THE DRAWINGS

FIG. 1 is a schematic representation of an embodiment of a method of screening DNA to identify mutations of interest.

FIGS. 2A, 2B and 2C are an embodiment of a technical report for AML generated using a disclosed method.

FIG. 3 is an embodiment of a variant report for AML generated using a disclosed method.

### DETAILED DESCRIPTION OF THE PREFERRED EMBODIMENTS

The foregoing aspects and many of the attendant advantages of this disclosure will become more readily apparent as the same become better understood by reference to the following detailed description.

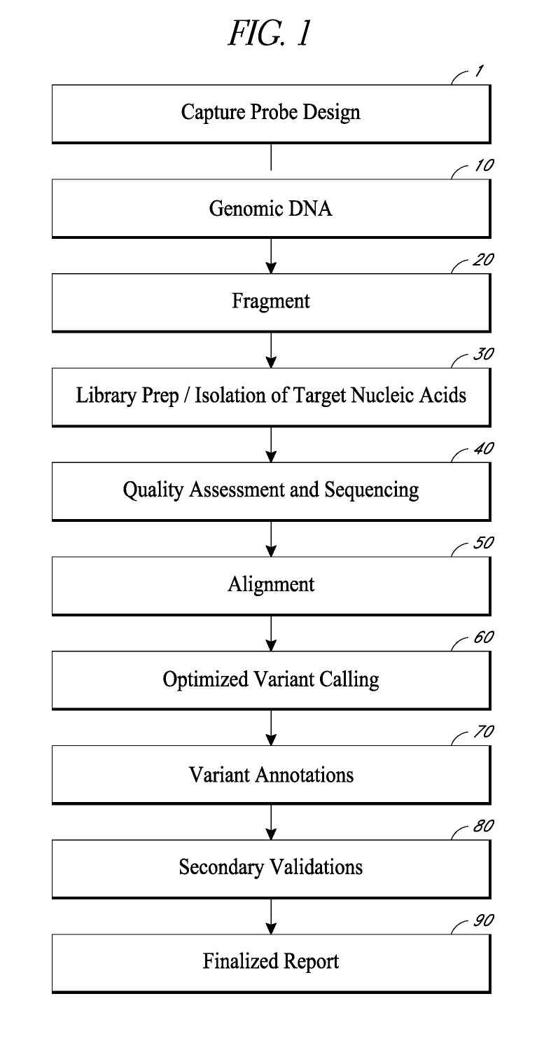

The technology described herein combines a series of discrete inventive steps and technologies that together comprise a method that brings unprecedented power to genomic screening, genetic analysis, and gene discovery. FIG. 1 provides a schematic of one embodiment of the disclosed invention. With reference to FIG. 1, a panel of capture probes are designed or selected 1 to capture target nucleic acids of interest from a sample. Nucleic acid which contains the target nucleic acids, for example genomic DNA 10, is isolated from a sample. The sample nucleic acid is fragmented, 20, and a library of fragmented nucleic acids for sequencing is prepared, (e.g. adding sequencing adaptors), and the target nucleic acids are isolated using the panel of capture probes 30. The quality of the isolated target nucleic acids is confirmed, and then they are sequenced 40. The sequence reads are aligned to a reference genome, 50, and variants are identified, 60. The variants are annotated, 70, validated, 80, and a final report is generated, 90, detailing the variants/mutations identified in the sample.

For example, in some embodiments, this next-generation sequencing method for the first time reliably detects novel structural mutations, translocations, and insertions and deletions. For example, in some embodiments the disclosed methods can detect large internal tandem duplications, or novel translocations, as well as identify the genomic breakpoint of novel translocations when only one of the two fusion partners is known or targeted. This is accomplished by employing a series of carefully selected capture probes to target genome-specific and disease-specific areas of target genes that harbor disease related somatic mutations, insertions/deletions or are involved in translocations.

In the preferred embodiment, the method pares down the entire genome to these discrete captured regions, leverages depth of sequence coverage in these target areas, enhances the sequencing data generated by employing methods that maximize sequencing read length, followed by analysis using a series of bioinformatic tools. By selectively restricting and defining the specific target areas that are captured and interrogated by sequencing (for example, drug and ligand target areas in proteins, and regulatory elements that might be involved with translocation partners) the depth of coverage and hence the sensitivity of this technology is enhanced. Sequencing read length in these targeted areas provides enhanced coverage of overlapping sequences that serve as the basis for bioinformatics algorithms that align the sequence reads to reference genomic databases. This allows the bioinformatics tools to more readily assign overlapping regions to large structural variants and translocations, even when the fusion partner is not known. In a preferred embodiment, the method combines the elements of 1) carefully defined gene- and disease-specific probe targeting; 2) capturing larger fragment sized genomic regions; 3) enhanced sequencing read depth; 4) longer sequencing read lengths, and 5) bioinformatics tools, to maximize the potential of this technology.

Embodiments of the disclosed invention can be used to identify some or preferably all somatic mutations and translocations in cancer. Somatic mutations may occur as a result of errors during DNA replication or through exposure to mutagens. Cancer cell genomes carry two types of somatic mutations: those mutations that confer a growth and survival advantage on the cell, and are positively selected for, and those that are not selected for. Thus, in addition to the difficulties in identifying somatic mutations generally, all somatic mutations are preferably detected to ensure identification of those mutations that drive cancerous growth.

Stratification of diagnosis, treatment, and/or prognosis of cancer is critical to elevating the state of clinical care for the cancer. The current application describes, in part, a precise mechanism for tracking the presence, emergence, and progression of mutations in nucleic acid sequences that drive cancer, such as AML. The ability to identify and then monitor these mutations with such precision enables faster more accurate diagnosis, facilitates proper patient stratification for enrollment in appropriate clinical trials, and may define the propensity for cancer. Furthermore, upon initiation of treatment, this technique can monitor the progression and effectiveness of therapy by monitoring the disappearance of mutated nucleic acid sequences that drive the cancer. Application of these methods will track the effectiveness of the therapy and provide guidance as to the prognosis of the patient. The disclosed techniques and methods can streamline diagnosis and improve the treatment of cancer, and will facilitate the timely development of more effective therapeutics.

By limiting the interrogation to genes affected by germline mutations, somatic mutations and translocation processes using the disclosed embodiments, one is able to more efficiently and reliably identify insertion site(s), ITD lengths, and allelic ratios for single nucleotide mutations, insertions, deletions and translocations, with increased sensitivity in detection of major and minor clonal populations. The disclosed embodiments resolve many of the limitations of current diagnostic and monitoring technologies and facilitate monitoring of minimal residual disease and clonal evolution during the course of treatment. This increased limit of detection provides a platform for the identification of some or all somatic mutations in cancer, ensuring the identification of those mutations that drive progression of the disease, many of which may be targets for therapy.

In some embodiments, the sensitivity of the disclosed methods can be increased by interrogating additional amounts of isolated nucleic acid from a greater number of cells. Sensitivity can also be increased by sequencing to a greater depth more of the enriched nucleic acids that are captured from a greater number of cells.

In some embodiments the disclosed methods are used in cancer to: stratify a range of patients presenting with different diseases or different subtypes of disease; be used to track one or more mutations directly for MRD analysis to track clones and subclones and better characterize the evolution of driver mutations during the course of treatment; and, even characterize cells lines in order to do a more comprehensive analysis of mutation status.

### DEFINITIONS

As used herein, “nucleic acid” or “nucleic acid molecule” can refer to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Nucleic acids can be either single stranded or double stranded.

As used herein, the terms “patient” and “subject” refer to a biological system from which a biological sample or biological data can be collected or to which a therapeutic agent can be administered. A patient can refer to a human patient or a non-human patient. Patients can include those that are healthy and those having a disease, such as cancer. Patients having a disease can include patients that have been diagnosed with the disease, patients that exhibit a set of symptoms associated with the disease, and patients that are progressing towards or are at risk of developing the disease.

### Capture Probe Design

Selection of Probes:

One aspect of embodiments disclosed herein is the selection or design of capture probes to use in the isolation of target nucleic acids which are subject to sequencing and analysis for mutations of interest. (FIG. 1, 1). In some embodiments the sub-genomic region(s) for interrogation are determined by reviewing the literature to identify in broad terms the mutation hotspots and translocation breakpoints that have been described for a specific disease. AML is one example provided herein, but the disclosed techniques are broadly applicable to virtually any disease state or process that might be impacted by genetic mutations or genomic architecture.

A variety of nucleic acid and protein databases are used to identify incompletely annotated or described nucleic acid sequences of both known and potential protein encoding subregions where regulatory proteins might bind as well as genomic regions that might encompass regulatory elements, such as enhancer or promoter regions. These regions typically correspond to only exon regions in many of the targeted genes, but may include intronic regions in other genes.

Next, the genomic coordinates that correspond to the genomic regions as well as regions flanking by several hundred to several thousand nucleotides are defined. The degree of resolution in the genomic region targeted by the capture probes is dependent on the confidence of the limit and scope of the region described. For many of the genes, there are not any specific hotspots for mutations, or there is uncertainty about the location of breakpoints at the genomic level, so the targeting of these genes can be more extensive than those genes wherein hotspots or specific mutations satisfy the analysis.

Extensive consideration of what regions of each gene should be included is given to the choice of each capture probe in the panel. For fusion genes, where intronic regions needed to also be included, a more involved analysis is required as these intronic regions can be incredibly large (for example, one intron in the PRPRT gene is over 300 kb in length). Therefore, diligent parsing of sequence area is necessary to maximize depth of coverage over the entire specialized gene panel. For example, AFF1 is a common gene involved in fusions. Its complete genomic sequence spans over 200 kb. However, the transcribed exons are limited to less than 10 kb. For this particular gene, narrowing down to the most relevant hotspot areas that are typically involved in fusions covers only 88,000 bp of the total exons and partial sequence of the introns, effectively not sequencing more than 110,000 bp of sequence.

Depth of Coverage:

The selection of capture probes used in the panel to capture cellular target nucleic acids is an important feature, as there is a limited band width (typically 3-4 MB using currently available sequencing systems) that sequencing provides. Therefore the precision and depth of sequencing is dependent on the choice of probes and the quantity of DNA that is captured and sequenced.

For example: whole genome sequencing of the 20,000+ genes and other DNA in the genome provides a depth of coverage of perhaps 1 or 2 reads for regions within a given gene; sequencing only exon regions increases this coverage to approximately 30-50×; limiting the selection still further to include just selected intron or exon regions within genes increases coverage even more.

For example, a 1-2 kb region within a genetic locus that often spans >1000 kb further increases the depth of coverage to say 1000 fold. Increasing probe baiting around difficult to detect regions involved in insertion and deletion mutations, and regions of complexity or high G:C content, can boost coverage even further to >5,000×. Examples are G:C rich regions of the gene CEBPA and the exon 14 and 15 regions of FLT3 involved in internal tandem duplication mutations.

Limiting capture probes to one or two specific exons of a single gene coupled with capturing and testing of multiple cell equivalents allows the depth of coverage to surpass 100,000×.

Accordingly, the disclosed methods are broadly applicable. Selection of the subset of regions of the 194 gene panel for AML described herein provides a depth of coverage in the 500×-1,500× range, with additional capture around certain critical regions so as to provide additional coverage 5,000×-10,000× around those regions that are either problematic from a hybridization perspective (e.g., CEBPA) or where additional coverage is desired or required so that the bioinformatics pipeline can place insertions and deletions with precision. In view of the disclosure herein, one of skill in the art can determine the desired level of coverage, and design a panel of capture probes to provide the desired level of coverage.

For example, capture probes for AML can include sequences which target the FLT3 gene, or a portion thereof. FLT3 (CD135) is a cytokine receptor in receptor tyrosine kinase class III, which is expressed on the surface of hematopoietic progenitor cells. FLT3 signaling, through homodimerization and autophosphorylation, impacts cell survival, differentiation, and proliferation. FLT3 signaling plays an important role in normal development of hematopoietic stem cells and is one of the most frequent mutations in AML. The AML capture probes could include regions to detect an ITD or length mutation, or other somatic mutations, such as single nucleotide variants, as discussed.

In a preferred embodiment, the capture probes are nucleic acids which hybridize to the target nucleic acids of interest and optionally include a moiety which assists in the isolation of the target nucleic acid when hybridized to the capture probe. The nucleic acid capture probe can comprise a DNA oligonucleotide, an RNA oligonucleotide, a combination of DNA/RNA oligonucleotide, or any related analogue (e.g., protein-nucleic acid hybrids) that has target specific hybridization properties, and may have a sense or antisense orientation. The capture probes are complementary to the target nucleic acid sequences. Preferably, they are 100% complementary, although capture probes that are, or are at least, 80%, 85%, 90%, 95%, 96%, 97%, 98%, or 99% complementary to the target nucleic acid sequence, or a range defined by any two of the preceding values, are contemplated. Complementarity can be measured over the entirety of the capture probe sequence. Capture probes can be used to enrich or isolate the target nucleic acids of interest by various methods known to those of skill in the art, including, but is not limited to, hybridization, immunoprecipitation, affinity purification, magnetic bead purification, and differential retention in solution, on a particle in suspension, or on a substrate.

Nucleic acid capture probes can be any length sufficient to provide the desired level of specificity necessary to capture the target nucleic acid. In a preferred embodiment, the nucleic acid capture probes are at least 15 nucleotides in length, preferably between about 25 and about 300 nucleotides in length. Also contemplated are nucleic acids capture probes that are, or are at least, 15, 20, 25, 30, 35, 40, 45, 50, 55, 60, 65, 70, 75, 80, 85, 90, 95, 100, 110, 120, 130, 140, 150, 175, 200, 225, 250, 275 or 300 nucleotides in length, or are a range defined by any of the preceding values. The nucleic acid capture probes used do not have to be of uniform length, but rather can vary in length depending on the number of nucleotides necessary to achieve the desired level of specificity to the target nucleic acid. In some embodiments, the nucleic acid capture probes specifically hybridize with the target nucleic acid sequence under stringent hybridization conditions, for example, either of the following: a) 6×SSC at about 45° C., followed by one or more washes in 0.2×SSC, 0.1% SDS at 65° C., and b) 400 mM NaCl, 40 mM PIPES pH 6.4, 1 mM EDTA, 50° C. or 70° C. for 12-16 hours, followed by washing.

The number of nucleic acid capture probe sequences in a panel is selected based on the factors discussed above, including the desired depth of coverage in view of the sequencing capacity of the sequencing method or instrument being used. In some embodiments, the number of capture probes sequences in a panel is between 10,000 and 200,000. Also contemplated are panels where the number of capture probe sequences is, or is at least, 1,000, 50,000, 100,000, 200,000, or a range defined by any two of the preceding values. Preferred numbers of nucleic acid capture probe sequences in a panel include from 1,000-50,000, 50,000-150,000, 100,000-200,000, or 150,000-300,000.

One or more moieties can optionally be included on a nucleic acid capture probe to facilitate later capture and/or identification of the target nucleic acid sequence. Examples include, but are not limited to an affinity probe (e.g. biotin), a photoreactive species, a hapten, a nucleic acid sequence or barcode, a fluorescent species, a protein, a carbohydrate, or another specific binding molecule or sequence for capture, identification, further amplification, enrichment or sequencing of the target nucleic acid.

Thus, in a preferred embodiment, target nucleic acid sequences hybridize with the nucleic acid capture probes, which are then located and/or captured using the included moiety. For example, the capture probe can be biotinylated and the subsequent probe-target complex can be captured with magnetic Streptavidin beads.

### Sample Preparation

A sample containing the cells of interest is obtained and the nucleic acids containing the target nucleic acids of interest are isolated from the sample by known methods. (FIG. 1, 10). The sample can be from a patient or subject suffering from a disease such as cancer, including but not limited to blood, bone marrow aspirate, or a tissue biopsy. Cultured cells could also be used. In some embodiments the isolated nucleic acid is genomic DNA, in others it is RNA or cDNA. In some embodiments, the sample is first treated to enrich the sample for a cell of interest, such as a cancer cell, using methods known in the art.

Following isolation of the nucleic acid from the biological sample, the nucleic acid is preferably fragmented. (FIG. 1, 20). This can be accomplished using methods known in the art, including but not limited to sonication, enzyme digestion, etc. The fragments are then purified to separate out preferred fragment sizes. The preferred average fragment size is ≧500 base pairs (bp) or nucleotides. In some embodiments, fragments smaller than about 150 bp nucleotides and larger than about 1500 bp/nucleotides in length are excluded. A preferred size range is from about 300 to about 700 bp/nucleotides, but contemplated average fragment sizes are, are at least, or are not more than, 150, 200, 300, 400, 500, 600, 700, 800, 900, 1000, 1100, 1200, 1300, 1400, 1500, 2000, 3000, 4000, 5000 or more bp/nucleotides, or a range defined by any two of the preceding values. Other average fragment sizes include 100-5,000, 200-1400, 200-1000, 200-800, 300-800, 300-1000, and 500-900 bp/nucleotides. In some embodiments, the fragments size or average fragments size listed herein is present in, or in at least 30%, 40%, 50%, 60%, 70% 80%, 90%, 95% or 100% of the total population, or a range defined by any of the preceding values, for example 40%-100%, 60%-100%. In some embodiments, the isolated nucleic acid is not fragmented, for example when the isolated nucleic acid is cDNA. Following isolation of fragments of the desired size, the fragmented nucleic acid sample is then optionally repaired (e.g., end repair and A-tail addition) and adaptor sequences are added to the fragments. (FIG. 1, 30). The adaptor sequences can be commercially available adaptors used in commercial sequencing methods, and can include identifiers (e.g. bar codes or other sequences) to allow identification of the source of the fragmented nucleic acid when nucleic acids from one or more samples are combined for sequencing or other subsequent method steps. Commercial adaptors and sequencing platforms include, for example, Illumina's MiSeq, HiSeq and Life Technologies', PGM platforms. KAPA Hyper Prep Kit (Kapa Biosystems, Wilmington, Mass.) is an example of a commercially available kit which includes end-repairing, A-tailing and adapter sequence ligation for use with Illumina's sequencing platforms. The adapter ligated fragments are then purified, and quantified. In a preferred embodiment, the purified fragmented nucleic acid library has an average size larger than 500 base pairs, and between 300-700 base pair fragments represent >40% of the total population.

If additional fragmented nucleic acids are desired, the nucleic acid can be amplified, either before fragmentation, before the adaptor is added, or, preferably, after the adapter is added. Amplification of these nucleic acids includes, but is not limited to, polymerase chain reaction, real time PCR, emulsion polymerase chain reaction, solid-phase amplification, rolling circle amplification, template mediated amplification, or isothermal amplification. The final concentration of fragmented nucleic acid is preferably ≧200 ng, more preferably >500 ng. The contemplated amount of fragmented nucleic acid is, or is at least, 50, 100, 150, 200, 250, 300, 350, 400, 500, 600, 700, 800, 900 or 1000 ng, or a range defined by any two of the preceding values.

### Isolation of Target Nucleic Acid

The capture probe panel discussed above is used to isolate the target nucleic acids of interest. (FIG. 1, 30). In a preferred embodiment, nucleic acid capture probes are hybridized to the fragmented nucleic acid libraries under conditions and for a time which allow for specific hybridization between the capture probe and its target nucleic acid. In some embodiments the hybridization is under stringent conditions. In some embodiments hybridization is at 47° C. for 2-72 hours. Where fragments from multiple samples are combined, equal amounts of each fragmented nucleic acid library from each sample are used to ensure equal numbers of sequencing reads from each component library. The captured target nucleic acids are recovered using known techniques, including but not limited to, immunoprecipitation, affinity purification, magnetic bead purification, and differential retention in solution, on a particle in suspension, or on a substrate.

The isolated target nucleic acids can be amplified and quantified using known techniques to ensure a sufficient quantity of target nucleic acids for the subsequent sequencing and/or analysis. One of skill in the art will recognize that the isolation of the target nucleic acids using the capture probes could be performed prior to the DNA repair and adaptor addition steps.

In a preferred embodiment, the target nucleic acid sequence is substantially free of other nucleic acid sequences following isolation using the capture probes. In some embodiments the target nucleic acid is, or is at least: 50% pure, more preferably 55% pure, more preferably 60% pure, more preferably 65% pure, more preferably 70% pure, more preferably 75% pure, more preferably 80% pure, more preferably 85% pure, more preferably 90% pure, more preferably 95% pure, more preferably 99% pure, or a range defined by any two of the preceding values.

As a non-limiting example, the isolation of target nucleic acid sequences is accomplished by capturing a subset of nucleic acid sequences characteristic of regions wherein variants, translocations or mutations stratify the diagnosis, treatment, or prognosis of AML. The subset of isolated AML target nucleic acids can also comprise an ITD or length mutation, or a somatic mutation, as discussed above.

### Sequencing

Once the target nucleic acids are isolated, the sample is sequenced. (FIG. 1, 40) The ability to both align sequences to a reference genome to identify large insertions and deletions and, perhaps more difficult, to identify fusion partners involved in gene translocations requires having sufficient flanking sequence outside of the captured target gene sequences with which to align sequences to other genetic regions within the genomic reference database. The longer the sequencing read the more flanking sequence is available for alignment. By adding specific size selection criteria such as longer shearing sizing and purification steps (e.g., at least 500 bp) to exclude shorter fragments, sequencing over longer fragments of DNA is increased. Additionally, novel targets can be identified by sequencing over adjoining fusion partners with these long sequencing reads, even though the actual capture probe set does not include that gene.

Translocations and large insertions/deletions (indels) are particularly difficult and, for the first time these structural mutations can be identified using the technology disclosed herein when capture probes and corresponding target nucleic acids are chosen correctly so as to encompass the regions required without diluting the band width required for sensitivity, when sufficient DNA is captured and sequenced to provide numbers of sequencing reads around the areas of importance, when the sequencing reads are of sufficient length to span large indels and translocation partners, and when the bioinformatic pipeline can interpret the resulting data and assign flanking sequences to novel genes—even when they reside on other chromosomes. In a preferred embodiment, the minimum concentration of isolated target nucleic acids utilized for the sequencing reaction is 1.5 nM.

In some embodiments the disclosed isolation and enrichment strategy provides clinical utility. Clinically actionable sensitivity for detection of minimal residual disease (MRD) is approximately 10⁻⁴; this sensitivity is possible with a read depth of coverage and tiling across the genes exceeding, to ensure the appropriate precision, 10,000 reads per sample; a read count of 1,000,000 generates sensitivity that approaches 10⁶. In a preferred embodiment, the read depth is, or is at least, 500×, 1000×, 5000×, 10,000×, 50,000×, or 100,000×, or a range defined by any two of the preceding values. In a preferred embodiment, the average length of the sequence reads of the isolated target nucleic acid fragments is, or is at least, 200, 300, 400, 500, 600, 700, 800, 900, 1000, 2000, 3000, 4000, 5000 or more nucleotides, or a range defined by any two of the preceding values. Average sequence read length can be 100-5,000, 200-1400, 300-1000, 300-700, 500-900, or 200-700 nucleotides. By varying conditions and different multiplex and sequencing strategies, the methods described herein are both scalable and flexible.

Sequencing of the isolated target nucleic acids includes, but is not limited to, Sanger sequencing, cyclic reversible termination, single-nucleotide addition, four-color sequencing, sequencing by ligation, pyrosequencing, single molecule sequencing, nanopore sequencing, sequencing by mass spectrophotometry, or real-time sequencing. Gnirke A. Melnikov A, Maquire J, Rogov P, LeProust E M, Brockman W, et al., Solution hybrid selection with ultra-long oligonucleotides for massively parallel targeted sequencing. Nat Biotechnol. 2009 February, 27(2):182-9 (incorporated herein by reference in its entirety), or chemistries that are compatible with existing instrumentation. Examples of combination sequencing chemistries and instrumentation approaches for next generation sequencing include, without limitation, Illumina's MiSeq, HiSeq and Life Technologies'. PGM platforms.

In some embodiments, next-generation sequencing is used to tile across at least one mutated region of nucleic acids wherein variants, translocations or mutations to achieve a depth to provide sufficient precision to identify that variant, translocation or mutation. This tiling strategy enables deep sequencing of a particular region of the genome, as opposed to more traditional genotyping methods, which probe for known or predicted sequences throughout an entire genome. Thus, tiling facilitates precise mapping of nucleic acid sequences implicated in disease, for example AML as described above, facilitating the identification of mutations and structural variants, identifying genetic breakpoints for translocations at the genomic DNA level, and identifying novel gene fusion partners. Data from these analyses can also be used to quantify the mutations relative to the wildtype or unmutated background sequences (allelic or mutation frequency), and to design more sensitive patient-specific MRD tests such as real-time tests for genomic DNA or cDNA.

In addition to amplification and measuring, the isolated target nucleic acids can also be imaged, wherein imaging includes, but is not limited to, capture of data generated by any method that differentiates normal genomic sequence from said nucleic acid sequences, including sequential assessment or measurement of single nucleotide or nucleotide analog incorporation, FRET signal production, or differential hybridization.

### Identification of Mutations

Following sequencing, the sequenced reads are aligned to a reference genome using one of any number of read mapping algorithms (eg: Novoalign, BWA, BFAST, Bowtie). (FIG. 1, 50). Aligned reads are then processed to improve mapping and to assess the quality of the sequencing and alignment. The aligned reads are evaluated to determine mutations/variants, including single nucleotide variants, insertions, deletions and structural variants such as translocations, using one or more of the following tools (VarScan, GATK, samtools, MuTect, BreakDancer, DELLY, Pindel). (FIG. 1, 60). Filters are used to eliminate low quality variants, and annotation methods are used to categorize the variants by their potential biological consequences. (FIG. 1, 70). A filtered subset of mutations with the highest likelihood of pathogenicity can then be manually curated to evaluate the potential impact of the mutation on the sample. (FIG. 1, 80).

Analyses can be conducted on the targeted nucleic acid sequences, for example, performing a bioinformatics analysis on the Internet accessible from a user computer. This bioinformatics analysis comprises identifying the mutant or identifying the mutant to wild type allelic ratios in nucleic acid sequences characteristic of regions that stratify the diagnosis, treatment, or prognosis of a disease such as cancer (e.g. AML), quantifying the mutant or quantifying the mutant to wild type allelic ratios in nucleic acid sequences characteristic of regions that stratify the diagnosis, treatment, or prognosis of the disease, and assigning specific intragenic locations nucleic acid sequences characteristic of regions that stratify the diagnosis, treatment, or prognosis of the disease.

Information gleaned from the compositions and methods disclosed herein can impact both the treatment protocols and patient outcomes in diseases characterized by genetic mutations such as cancer. The resulting data regarding mutations present in the sample can be used for various purposes, including diagnosis or prognosis of disease, monitoring patient care or for the development of new screening or diagnostic tools, MRD tests, and use of new mutations or patient-specific mutations for use as new biomarkers.

The treatment of the disease can be modified by administering a treatment or agent that modulates or targets the activity or expression of at least one gene identified within said nucleic acid sequences that comprise variants, translocations or mutations that stratify the diagnosis, treatment, or prognosis of the disease. Furthermore, the treatment of the disease can be monitored by examining the subset of isolated target nucleic acid sequences identified either by subsequent testing using this technology, or by using sequence information obtained from this technology to design other MRD approaches, such as real-time PCR. In other embodiments, the subset of targeted nucleic acid sequences can be correlated with the activity of a drug targeting at least one expressed biological product thereof. Still further, the efficacy of treatment may be determined by examining the subset of isolated target nucleic acid sequences identified either by subsequent testing using this technology, or by using sequence information obtained from this technology to design other MRD approaches, such as real-time PCR with the level of expression of another gene or product of another gene.

In some embodiments a result can be generated wherein the result consists of a report identifying at least one variant, translocation or mutation that stratify the diagnosis, treatment, or prognosis of a disease. (FIG. 1, 90). This result can be provided by electronic, web-based, or paper means to, for example, a patient, another person or entity, a medical power of attorney, a caregiver, a physician, a health care practitioner, oncologist, a hospital, clinic, third-party payor, insurance company, pharmaceutical company, or government office.

After reading this description it will become apparent to one skilled in the art how to implement the invention in various alternative embodiments and alternative applications. For example, a preferred embodiment is illustrated in FIG. 1 and described above, wherein the sample DNA is isolated from sample, the sample is fragmented and size selected, adaptors are added and the resulting nucleic acids are amplified prior to using the capture probe panel to isolate target nucleic acids. However, one of skill in the art will recognize that many of these steps can be carried out in a different order, and some steps may not be necessary at all. For example, the panel of capture probes does not have to be designed or selected before the fragmented library is prepared. As another non-limiting example, the capture probes could be used to isolate fragmented target nucleic acids prior to the size selection, adaptor addition and amplification. Applicants specifically contemplate that the values for different parameters specified throughout the disclosure can be selected and combined even where specific combinations of values for parameters are not specifically disclosed. As a non-limiting example, Applicants contemplate selection of a value for the number of capture probes from any of the values or ranges disclosed for that parameter, as well as selection of a read depth from any of the values or ranges disclosed for that parameter, such that a method having the selected number of capture probes and the selected read depth is contemplated. The following examples are non-limiting examples of embodiments of the invention disclosed herein.

### EXAMPLES

### Example 1

### Design of Capture Probes for Screening of AML

By extensive curation of the literature on genes known to or suspected to impact development of AML, we have compiled a list of 194 relevant genes. The gene list is broken down into 3 subsets based on (1) NCCN/ELN guidelines; (2) those genes most commonly rearranged in AML that include breakpoints with their intronic structures (3) coding sequences or exons of genes suspected to be involved in the etiology of AML development (see Table 1). One major literature source was The Cancer Genome Atlas that recently characterized 200 AML samples. Based on the somatic mutation frequency rate for AML, it is calculated that 95% of all the mutations that are involved in AML have now been identified. The literature that was used for compiling this panel includes well over 300 publications.

TABLE 1
NCCN/ELN Guidelines
Structural Rearrangements: Inv(16) t(16;16) t(8;21) t(15;17) +8 t(9;11) −5 5q-−7 7q-11q23 inv(3)
t(3;3) t(6;9) t(9;22)
[These regions also include genes from the ‘Other Fusions/Gene rearrangements’ below]
Genes: CEBPA DNMT3A FLT3 IDH1 IDH2 KIT NPM1
[Including 5′UTRs, Exons, Non-coding Exons, and 3′UTRs]
Other Fusions/Gene rearrangements (36 Genes)
[Including 5′UTRs, Exons, Recombination Intron Breakpoint Hotspots, Non-coding Exons, and
3′UTRs]
ABL1 AFF1 BCR CBFB CREBBP DEK EIF4E2 ELL ETV6 GAS6 GAS7 GPR128 KAT6A KAT6B KMT2A
MECOM MKL1 MLLT10 MLLT1 MLLT3 MLLT4 MYH11 NSD1 NUP214 NUP98 PICALM PML RARA
RBM15 RPN1 RUNX1 RUNX1T1 SEPT5 SET TFG TMEM255B
Other Genes (151)
[Including 5′UTRs, Exons, Non-coding Exons, and 3′UTRs]
ABCC1 ACVR2B ADRBK1 AKAP13 ANKRD24 ARID2 ARID4B ASXL1 ASXL2 ASXL3 BCOR BCORL1
BRINP3 BRPF1 BUB1 CACNA1E CBL CBX5 CBX7 CDC73 CEP164 CPNE3 CSF1R CSTF2T CTCF CYLD
DCLK1 DDX1 DDX23 DHX32 DIS3 DNAH9 DNMT1 DNMT3B DYRK4 EED EGFR EP300 EPHA2 EPHA3
ETV3 EZH2 FANCC GATA1 GATA2 GFI1 GLI1 HDAC2 HDAC3 HNRNPK HRAS IKZF1 JAK1 JAK2 JAK3
JMJD1C KDM2B KDM3B KDM6A KDM6B KMT2B KMT2C KRAS MAPK1 METTL3 MST1R MTA2 MTOR
MXRA5 MYB MYC MYLK2 MYO3A NF1 NOTCH1 NOTCH2 NRAS NRK OBSCN PAPD5 PAX5 PDGFRA
PDGFRB PDS5B PDSS2 PHF6 PKD1L2 PLRG1 POLR2A PRDM16 PRDM9 PRKCG PRPF3 PRPF40B
PRPF8 PTEN PTPN11 PTPN14 PTPRT RAD21 RBBP4 RBMX RPS6KA6 SAP130 SCML2 SETBP1 SETD2
SF1 SF3A1 SF3B1 SMC1A SMC3 SMC5 SMG1 SNRNP200 SOS1 SPEN SRRM2 SRSF2 SRSF6 STAG2
STK32A STK33 STK36 SUDS3 SUMO2 SUPT5H SUZ12 TCF4 TET1 TET2 THRB TP53 TRA2B TRIO TTBK1
TYK2 TYW1 U2AF1 U2AF1L4 U2AF2 UBA3 WAC WAPAL WEE1 WNK3 WNK4 WT1 ZBTB33 ZBTB7B
ZRSR2
MicroRNA (2)
[Sequence only]
Mir-142 Mir-155
Total: Genes 194 + 2 microRNA

A panel of approximately 196,000 unique capture probes, each between about 20-200 nucleotides in length, targeted to the genes 194 AML genes listed in Table 1 was designed. The capture probes were directed to portions of the 194 genes identified as involved in, or likely to be involved in, a nucleic acid mutation, such as a single nucleotide variant, an insertion or deletion (InDel) or translocation. The sequences of the capture probe panel are disclosed in the Sequence Listing submitted in the priority document U.S. Patent Application 61/900,728, filed on Nov. 6, 2013, which is incorporated herein by reference.

### Example 2

### Identification of Mutations in AML Cells

Genomic DNA isolated from a mixture of AML cells was fragmented into average sizes of 700 basepairs (bp) fragments using a Covaris ultrasonicator (Covaris, Woburn, Mass.). DNA fragments were then purified using Ampure XP (Beckman Coulter, Brea, Calif.) following manufacture suggested procedures. This step is important to separate out the longer, preferred fragment sizes (700 bp), from the smaller, less preferred fragment sizes (below 150 bp, and greater than 1500 bp). Longer, purified DNA fragments were analyzed by a LabChip (PerkinElmer, Waltham, Mass.) to ensure that the fragments size distribution primarily fell in the range of 500-900 bp. The DNA was then repaired, and adaptor sequences (commercially available) were added to identify separate DNA samples from one another in subsequent steps (called multi-plexing). End-repairing, A-Tailing, and Adapter ligation of the DNA library was constructed using KAPA Hyper Prep Kit (Kapa Biosystems, Wilmington, Mass.) by following manufacture suggested procedures. After this construction, the Adapter ligated fragments were purified using Ampure XP by following manufacture suggested procedures.

Adaptor ligated fragments were quantified using KAPA Hyper Prep Kit by following manufacture suggested procedures, and amplified DNA was again purified using Ampure XP by following manufacture suggested procedures. To ensure that the concentration, size distribution, and quality of the fragmented DNA library were sufficient, the Kapa Library Quantification Kit (Kapa Biosystems, Wilmington, Mass.) and HT DNA HiSens Reagents for the LibChip GX (PerkinElmer, Waltham. Mass.) were employed.

Hybridization of pre-capture fragmented DNA library using the approximately 196,000 capture probes from Example 1 followed. To obtain equal numbers of sequencing reads from each component libraries in the multiplex DNA library, equal amounts of each independently amplified DNA library were normalized for the hybridization. The hybridization samples were incubated at 47° C. for 2-72 hours. The captured DNA library of target nucleic acids was recovered using Nimblegen Hybridization and Wash Kit (Roche NimbleGen, Madison, Wis.) by following manufacture suggested procedures. The post-capture DNA target nucleic acid library was amplified and quantified using KAPA HiFi Library Amplification Kit (Kapa Biosystems, Wilmington. Mass.) by following manufacture suggested procedures. The captured-amplified target nucleic acid DNA library was purified using Ampure XP by following manufacture suggested procedures.

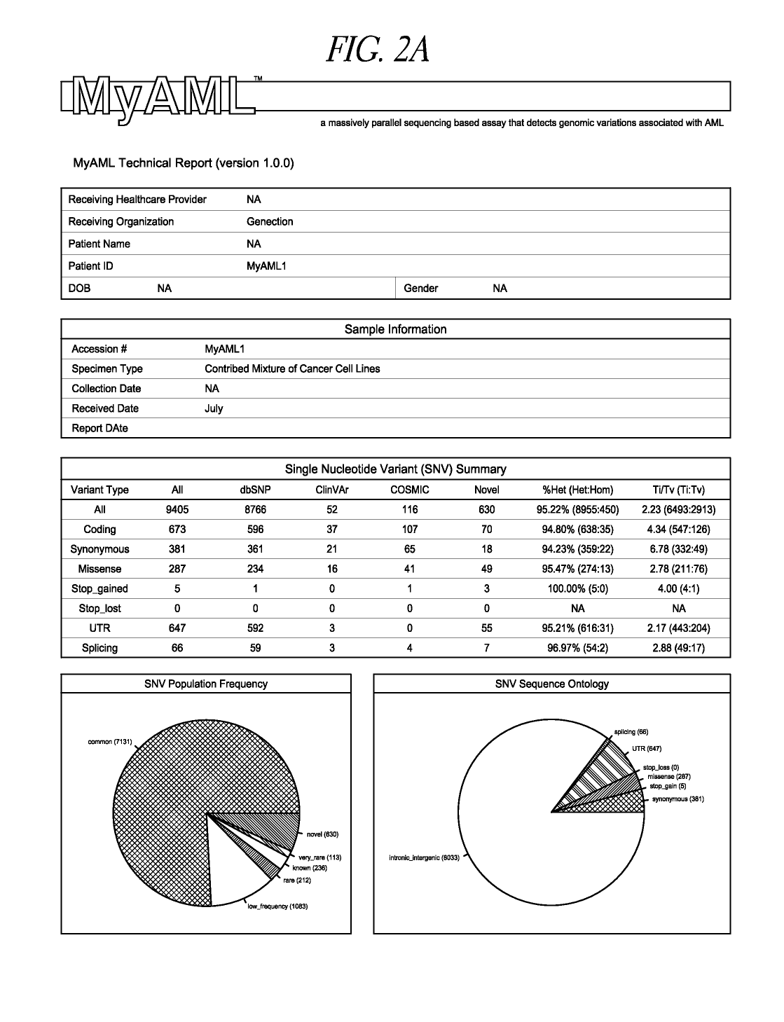

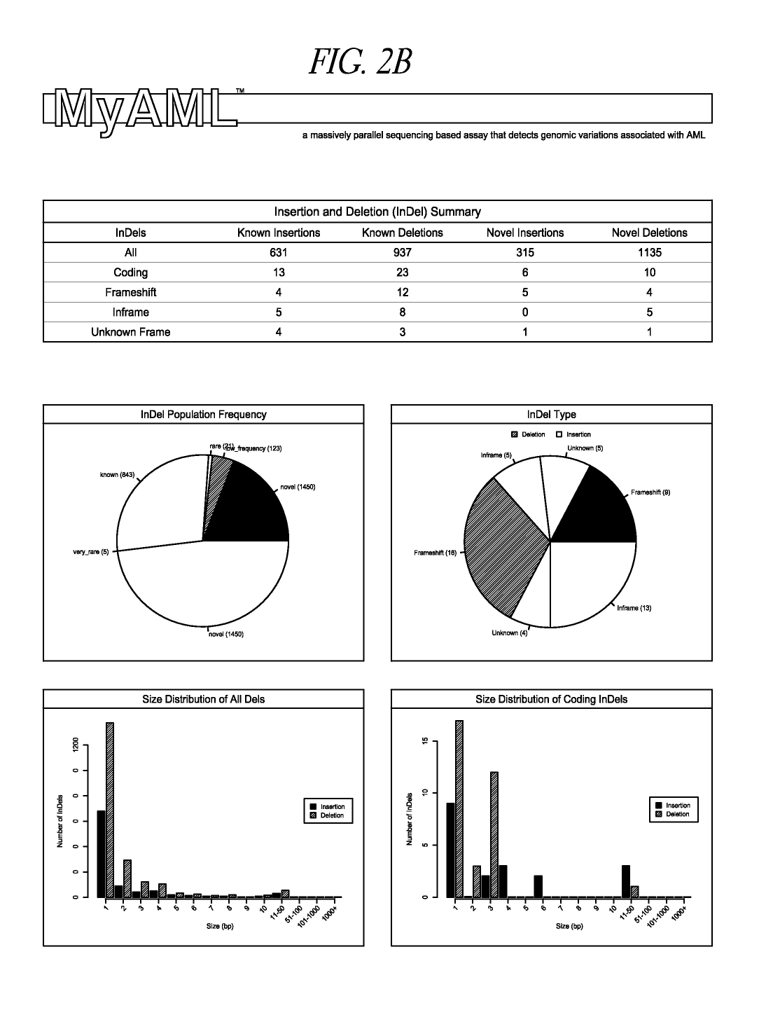

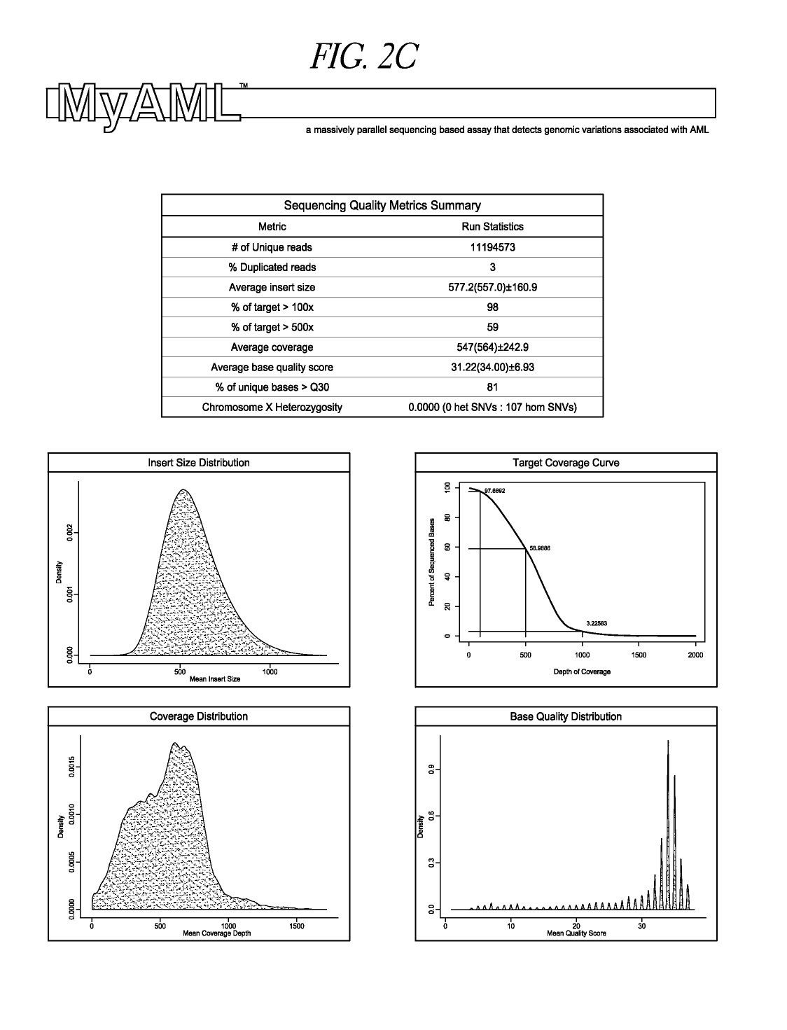

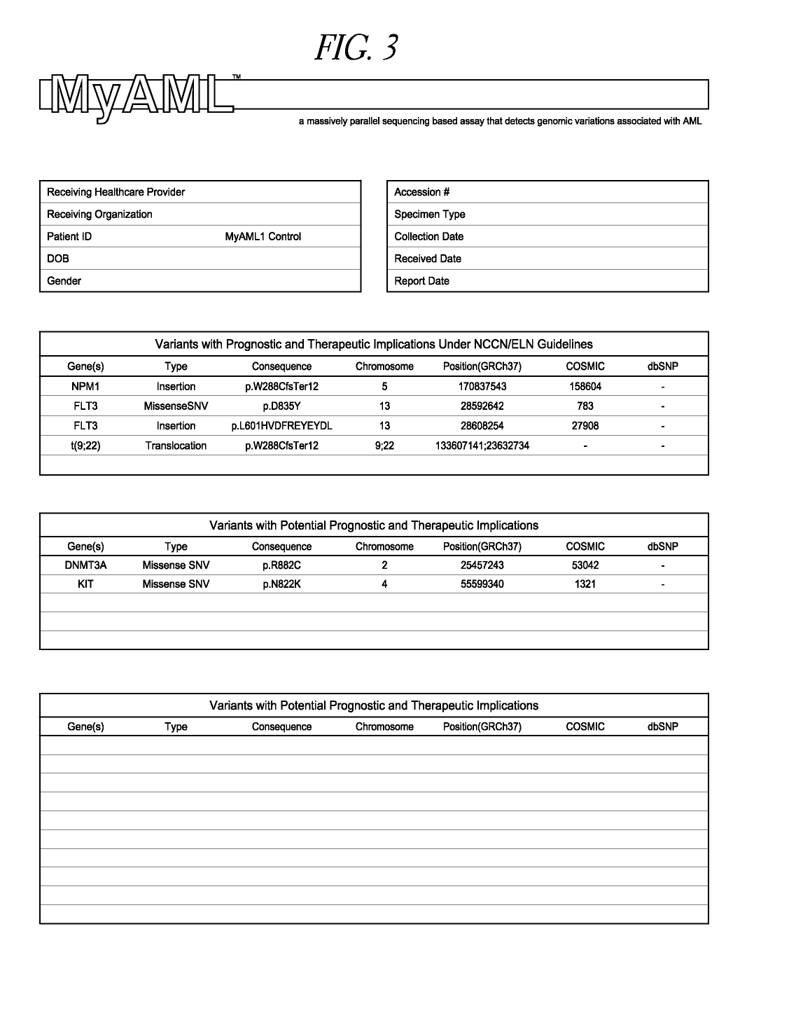

The final concentration of the target nucleic acid DNA library was determined using Kapa Library Quantification Kit and HT DNA HiSens Reagents for the LibChip GX. The Library was then loaded and sequenced on MiSeq, (Illumina, San Diego, Calif.) and samples were sequenced, generating paired reads that were stored in .fastq format. Sequenced reads were then aligned to a reference genome using one of any number of read mapping algorithms (eg: Novoalign, BWA, BFAST, Bowtie). Aligned reads were then processed to improve mapping and to assess the quality of the sequencing and alignment. Aligned reads were then evaluated to determine mutations/variants, including single nucleotide variants, insertions, deletions and structural variants using one or more of the following tools (VarScan, GATK, samtools. MuTect, BreakDancer, DELLY, Pindel). Filters were then applied to remove low quality variants and annotation methods were used to categorize the variants by their potential consequences. Finally, after filtering variants to a subset containing mutations with the highest likelihood of pathogenicity, the final variant set was manually curated to evaluate the potential impact of the variant on the sample. An exemplary technical report is shown in FIGS. 2A-2C, which includes the raw numbers of mutations/variants found. FIG. 3 is an exemplary variant report, listing mutations/variants with prognostic and therapeutic implications.

After reading this description it will become apparent to one skilled in the art how to implement the invention in various alternative embodiments and alternative applications. However, all the various embodiments of the present invention will not be described herein. It is understood that the embodiments presented here are presented by way of an example only, and not limitation. As such, this detailed description of various alternative embodiments should not be construed to limit the scope or breadth of the present invention as set forth herein.

What is claimed is:

1. A method of screening a nucleic acid sample for mutations comprising:
(a) obtaining a nucleic acid sample;
(b) fragmenting the nucleic acid sample;
(c) contacting the fragmented nucleic acid sample with a panel of capture probes, wherein the panel of capture probes specifically capture targeted nucleic acid fragments which are identified as having or likely having a mutation;
(d) isolating the targeted nucleic acid fragments captured by the panel of capture probes;
(e) sequencing the isolated targeted nucleic acid fragments; and
(f) analyzing the sequences of the isolated targeted nucleic acid fragments to identify mutations with prognostic and/or therapeutic significance.

2. The method of claim 1, further comprising:
(b′) adding adaptor nucleic acids to the fragmented nucleic acids.

3. The method of any of claim 1 or 2, wherein the panel of capture probes comprise a plurality of nucleic acids comprising at least 1,000 unique nucleic acid sequences, at least 10,000 unique nucleic acid sequences, at least 100,000 unique nucleic acid sequences, at least 150,000 unique nucleic acid sequences, or at least 200,000 unique nucleic acid sequences.

4. The method of any of claims 1-3, wherein the nucleic acid capture probes are 20-200 nucleotides in length, or 50-200 nucleotides in length, or 20-150 nucleotides in length.

5. The method of any one of claims 1-4, wherein the nucleic acid capture probes have a nucleic acid sequence which is complementary to the targeted nucleic acid fragments, wherein the complementarity is at least 80% complementarity, 90% complementarity, 95% complementarity, or 100% complementarity.

6. The method of any of claims 1-5, further comprising
(b″) selecting the nucleic acid fragments to select nucleic acid fragments of 100-5,000 nucleotides in length, 200-1400 nucleotides in length, or 300-900 nucleotides in length, or 300-700 nucleotides in length.

7. The method of any of claims 1-6, wherein the isolated targeted nucleic acid fragments have an average length of 100-5,000 nucleotides in length, 200-1400 nucleotides in length, or 300-900 nucleotides in length, or 300-700 nucleotides in length.

8. The method of any of claims 1-7, wherein the sequencing of the isolated target nucleic acid fragments is at a read depth of at least 500×, at least 1000×, at least 10,000×, or at least 100,000×.

9. The method of any of claims 1-8, wherein the average length of the sequence reads of the isolated target nucleic acid fragments is at least 500 nucleotides, or at least 600 nucleotides, at least 700 nucleotides, or at least 1,000 nucleotides.

10. The method of any of claims 1-9, wherein the analyzing comprises aligning the sequences of the isolated targeted nucleic acid fragments to a reference sequence.

11. The method of any of claims 1-10, wherein the nucleic acid sample is isolated from a biological sample.

12. The method of any of claims 1-11, wherein the nucleic acid sample is isolated from a sample comprising cancer cells.

13. The method of any of claims 1-12, wherein the target nucleic acids are from genes identified as having a mutation in a cancer cell.

14. The method of any of claims 1-13, wherein the target nucleic acids are from genes identified in a public database as having a mutation in a cancer cell.

15. The method of any of claims 1-14, wherein the identified mutation is used for diagnostic, prognostic, or treatment purposes.

16. The method of any of claims 1-15, wherein the sample is from a patient, and the identified mutation is used for diagnostic, prognostic, or treatment purposes.

17. The method of any of claims 1-16, wherein the mutation is selected from the group consisting of a single nucleotide variant, an insertion, a deletion or a translocation.

18. The method of any of claims 1-17, wherein step (b′) is before step (c).

19. The method of any of claims 1-17, wherein step (b′) is after step (c).

20. The method of any of claims 1-19, wherein step (b″) is before step (c).

21. The method of any of claims 1-19, wherein step (b″) is after step (c).

22. A panel of nucleic acid capture probes comprising a plurality of nucleic acids, wherein the nucleic acids are 20-200 nucleotides in length, wherein the nucleic acids comprise at least 1,000 unique nucleic acid sequences, and wherein the nucleic acid sequences are complementary to target nucleic acids that are identified as having or likely having a mutation.

23. The panel of claim 22, wherein the mutation is selected from the group consisting of a single nucleotide variant, an insertion, a deletion or a translocation.

24. The panel of any of claims 22-23, wherein the target nucleic acids are from genes identified as having a mutation in a cancer cell.

25. The panel of any of claims 22-24, wherein the target nucleic acids are from genes identified in a public database as having a mutation in a cancer cell.

26. The method of panel of any of claims 1-25, wherein the panel of capture probes comprise at least 10,000 unique nucleic acid sequences complementary to at least 30 genes selected from Table 1.
